(19) United States
(12) Patent Application Publication
(10) Pub. No.: US 2024/0075195 A1
(43) Pub. Date: Mar. 7, 2024

(54) Device for Establishing the Venous Inflow to a Blood Reservoir of an Extracorporeal Blood Circulation System

(71) Applicant: LivaNova Deutschland GmbH, Munich (DE)

(72) Inventors: Erwin Knott, Poing (DE); Johann Schreyer, München (DE)

(73) Assignee: LivaNova Deutschland GmbH, Munich (DE)

(21) Appl. No.: 18/506,414

(22) Filed: Nov. 10, 2023

Related U.S. Application Data
(63) Continuation of application No. 17/554,393, filed on Dec. 17, 2021, now Pat. No. 11,844,892.
Application No. 17/554,393 is a continuation of application No. 16/180,870, filed on Nov. 5, 2018, now Pat. No. 11,229,729.
Application No. 16/180,870 is a division of application No. 13/322,844, filed on Feb. 7, 2012, now abandoned.

(30) Foreign Application Priority Data
May 29, 2009 (DE) 102009026592.9

(51) Int. Cl. A61M 1/36

(52) U.S. Cl. CPC A61M 1/3621; A61M 1/3667; A61M 1/3627; A61M 1/3663; A61M 2205/3389

(57) ABSTRACT

A device for establishing venous inflow to a blood reservoir of an extracorporeal blood circulation system includes a restricting unit for gradually closing a venous inflow line and a vacuum unit for supplying vacuum to the blood reservoir. The device includes a control unit that supplies a first actuating signal to the restricting unit for restricting venous inflow to the blood reservoir and supplies a second actuating signal to the vacuum unit for establishing a degree of vacuum within the blood reservoir.

### CROSS REFERENCE TO RELATED APPLICATIONS

This application is a continuation of U.S. patent application Ser. No. 17/554,393, filed Dec. 17, 2021, which is a continuation of U.S. patent application Ser. No. 16/180,870, filed Nov. 5, 2018, which is a division of U.S. application Ser. No. 13/322,844, filed Feb. 7, 2012, which is a National Phase Application of PCT/EP2010/055522, filed pursuant to 35 U.S.C. § 371, which claims priority to DE 102009026592.9, filed May 29, 2009, the entirety of which are incorporated herein by reference.

### TECHNICAL FIELD

The invention relates to a device for establishing venous inflow to a blood reservoir in an extracorporeal blood circulation system.

### BACKGROUND

An extracorporeal blood circulation system often includes, in addition to other components, a venous inflow line from a patient to a blood reservoir, for example a venous cardiotomy reservoir for blood and/or priming solution, and an arterial inflow line from the reservoir to the patient. In order to convey blood from the patient into the reservoir, the reservoir is located at a lower level than the patient so that drainage can already occur by means of gravity. Using a blood pump, usually a roller pump, the blood is conveyed out of the reservoir through the arterial inflow line and is delivered to the patient.

So that the venous inflow to the reservoir occurs to a sufficient extent, large tube cross-sections are required in the case of pure gravitational drainage, which is problematic in respect of the strain on the patient which is associated with the drainage line. Furthermore, pure gravitational drainage is unfavorable as regards the ability to influence the venous inflow amount since in order to do so, the difference in height between the reservoir and the patient would have to be altered while maintaining the extracorporeal blood circulation.

As is described, for example, in WO 00/44415 A, it has already been proposed to apply a vacuum to the reservoir in order to reduce the cross-section of the drainage line and to be able to influence the venous inflow. In this regard, WO 00/44415 A discloses a vacuum regulator that simplifies management of the vacuum by the user so as to increase patient safety during an operation. The reason for this is that a reliable regulation of the vacuum first of all enables a simple adjustment of the vacuum by a user and thus a simple adjustment of the venous inflow. A reliable regulation of the vacuum also prevents a vacuum which is too high and which presents a danger to the patient, whereby it must be noted that the withdrawal of the blood from the reservoir using the blood pump which arterially supplies the blood to the patient contributes to the creation and/or increase of the vacuum. In WO 00/44415 A, previously known solutions are referred to as being insufficient and an independent device for regulating the vacuum is described, which reliably regulates the vacuum in the reservoir and optionally also reduces it should this be necessary.

When supplying blood from the reservoir to the patient, care must be taken to ensure that no more blood is removed from the reservoir than is present therein or is supplied thereto. The user must therefore make sure that there is a sufficient minimum amount in the reservoir and additionally coordinate the inflow and outflow such that sufficient amounts are built up in the reservoir but are also supplied to the patient since a physiologically sufficient supply of the patient must always be ensured in an extracorporeal blood circulation system. The user may thereby, for example, increase or reduce the delivery rate of the roller pump or he may reduce the line cross-section of the supply line by means of clamping and/or increase it again. In addition, the user must pay attention to the amount of drained blood and suitably adjust the vacuum in the reservoir—even in the aforementioned vacuum regulator. The user must meet this requirement in an environment which furthermore compels him, in all of his actions, to pay attention to the surgical operation supported by the extracorporeal circulation. Overall, the adjustment of the venous inflow amount to the reservoir of an extracorporeal blood circulation system therefore occurs in a stressful working environment, and thus the previous technical solutions for supporting the user in the adjustment of the venous inflow amount to the reservoir must be regarded as sub-optimal.

Against this background, the technical problem to be solved by the invention is to specify a device for establishing the venous inflow to a blood reservoir of an extracorporeal blood circulation system, which further simplifies the management of the extracorporeal blood circulation system for the user.

### SUMMARY

A device as according to the invention for establishing the venous inflow to a blood reservoir of an extracorporeal blood circulation system, which includes a venous inflow line from a patient to the reservoir and an arterial inflow line from the reservoir to the patient, further includes, in addition to a restricting unit for gradually closing, in particular clamping, the venous inflow line in order to restrict the venous inflow amount to the reservoir and a vacuum unit for applying a vacuum to the reservoir in order to increase the venous inflow amount to the reservoir, a control means which supplies to the restricting unit a first actuating signal for establishing the degree of closure of the venous inflow line in order to determine the extent of the restriction of the venous inflow amount to the reservoir, and which supplies to the vacuum unit a second actuating signal for establishing the amount of vacuum in the reservoir in order to determine the extent of the increase in the venous inflow amount to the reservoir, and which includes a single operating element for setting of the amount of venous inflow to the reservoir by a user.

By providing a single operating element for setting of the amount of venous inflow to the reservoir by a user, the user is given the opportunity in a simple manner to determine the venous inflow to the reservoir at, above and below a basic value by a single adjustment process. The basic value is set by restricting conveyance in the case of venous inflow amounts below this value and by actively supporting conveyance of the inflow amount, which is otherwise conveyed and defined by gravity, in the case of venous inflow amounts above this value. As a result of the configuration of the control means according to the invention, it is achieved that the device according to the invention appropriately adjusts and regulates the venous inflow. It should be noted that the single operating element described above and in the following is solely for the function of setting the amount of venous inflow to the reservoir by a user. The control means can additionally include further operating elements, such as, for example, an on/off switch, a brightness regulator for optionally provided displays, a selector switch for activating/deactivating an alarm, etc. However, according to some embodiments of the invention, only one single operating element is provided for setting of the amount of venous inflow to the reservoir by a user, said element allowing setting of the venous inflow both at above and below a basic value.

In some embodiments, the control means includes a display device for visual display of a display value corresponding to the amount of venous inflow. By means of this display, an indication of value as regards the venous inflow, the vacuum in the reservoir and/or the amount of blood stored in the reservoir can be displayed to the user.

In some embodiments, the vacuum unit is connected to a vacuum source via a line or alternatively or additionally includes an integrated vacuum source, in particular a pump.

In some embodiments, a vacuum sensor for detecting the vacuum in the reservoir is provided, which is connected to the control means for providing a corresponding measuring signal.

In some embodiments, a level sensor for detecting the level of blood present in the reservoir is provided, which is connected to the control means for providing a corresponding measuring signal.

In some embodiments, an arterial inflow amount sensor for detecting the inflow amount conveyed in the arterial inflow line is provided, which is connected to the control means for providing a corresponding measuring signal.

In some embodiments, the control means generates a third actuating signal which establishes the delivery rate of a blood pump such that the arterial inflow amount can at least be influenced.

In some embodiments, the single operating element is configured for specifying an amount of blood to be stored in a reservoir so that the venous inflow to the reservoir can be set in this manner.

In some embodiments, the vacuum unit includes a safety device which, upon actuation by the control means or if control should fail, interrupts the application of a vacuum to the reservoir and establishes atmospheric pressure in the reservoir.

### BRIEF DESCRIPTION OF THE DRAWINGS

The invention will be described in more detail in the following with reference to the drawings, in which:

FIG. 1 shows a view of the basic structure of an extracorporeal blood circulation system having a device according to a first embodiment of the invention;

FIG. 2 shows a view of the basic structure of an extracorporeal blood circulation system having a device according to a second embodiment of the invention;

FIG. 3 shows a view of the basic structure of an extracorporeal blood circulation system having a device according to a third embodiment of the invention; and

FIG. 4 shows a view of the basic structure of an extracorporeal blood circulation system having a device according to a fourth embodiment of the invention.

### DETAILED DESCRIPTION

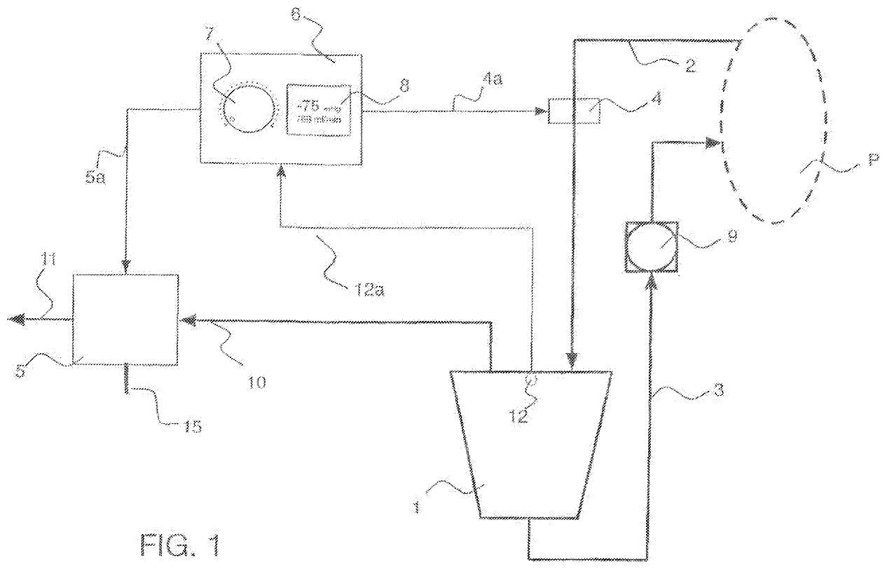

As is shown in FIG. 1 by means of a first embodiment of a device according to the invention, an extracorporeal blood circulation system basically includes, in addition to a reservoir 1, a venous inflow line 2 from a patient P to the reservoir 1 and an arterial inflow line 3 from the reservoir 1 to the patient P. In order to supply blood from the reservoir 1 to the patient P, a pump 9, such as a roller pump or a centrifugal pump, is provided on or in the arterial inflow line 3. As is apparent from FIG. 1, the reservoir 1 is located at a lower level than the patient so that a venous inflow to the reservoir is already possible owing to gravity alone, the extent of which also depends, however, on the cross-section of the tube used in the venous inflow.

In order to restrict the venous inflow to the reservoir 1, the first embodiment of the invention as described herein includes a restricting unit 4, by which the venous inflow line 2 can be gradually closed, for example by clamping or squeezing. For this purpose, the restricting unit 4 includes an electromagnetically, pneumatically or hydraulically-operated clamp which is not shown in detail in FIG. 1. The restricting unit 4 is arranged on the venous inflow line 2 and in some embodiments acts externally on the tube without coming into contact with the blood.

In order to increase the venous inflow to the reservoir 1, the first embodiment of a device according to the invention includes a vacuum unit 5 for applying a vacuum to the reservoir 1. For this purpose, the vacuum unit 5 is connected via a vacuum line 10 to the reservoir 1 which is accordingly designed so as to be pressure-tight. If the vacuum unit 5 is itself not configured for generating the vacuum, for example by integration of a suitable pump, the vacuum unit 5, as is the case in the first embodiment shown in FIG. 1, is connected to a vacuum source via a vacuum source line 11. Suitable vacuum sources are generally available in the form of stationary installations in hospitals where the device according to the invention is primarily used.

According to the invention, the setting of the venous inflow by the user occurs in a simple and comfortable yet also safe manner via a control means 6 which is shown in FIG. 1. The control means 6 supplies to the restricting unit 4, via a first actuation line 4a, a first actuating signal for establishing the degree of closure of the venous supply line 2 in order to carry out restriction of the venous inflow amount to the reservoir. The control means 6 furthermore supplies to the vacuum unit 5, via a second actuation line Sa, a second actuating signal for establishing the amount of vacuum in the reservoir 1 in order to carry out an increase in the venous inflow amount to the reservoir.

According to the invention, the control means 6 includes a single operating element 7 for setting of the amount of venous inflow to the reservoir by a user of the device. Solely by actuating this single operating element 7, the user can set the venous inflow amount to the reservoir 1 and thereby undertake both an increase to beyond the amount which is essentially determined by the gravitational drainage and the cross-section of the tube used on the venous side, as well as a reduction to below this value. The control means 6 according to the invention converts the setting carried out by the user by means of the single operating element 7 into a corresponding actuation of the restricting unit 4 or the vacuum unit 5 so as to thereby cause a reduction of the venous inflow amount by closing (clamping) the venous inflow tube line 2 or an increase in the venous inflow amount by creating a vacuum in the reservoir 1.

As is shown by FIG. 1, the first embodiment includes a pressure sensor 12, which is arranged on or in the reservoir 1 so as to detect the vacuum in the reservoir 1 and provide a corresponding first measuring signal. The measuring signal of the vacuum sensor 12 is supplied via a first measuring signal line 12a to the control means 6, which can carry out regulation of the vacuum taking into account this measuring value.

The control means 6 according to the first embodiment furthermore includes a display 8 which indicates to the user the set venous inflow amount and/or the set vacuum.

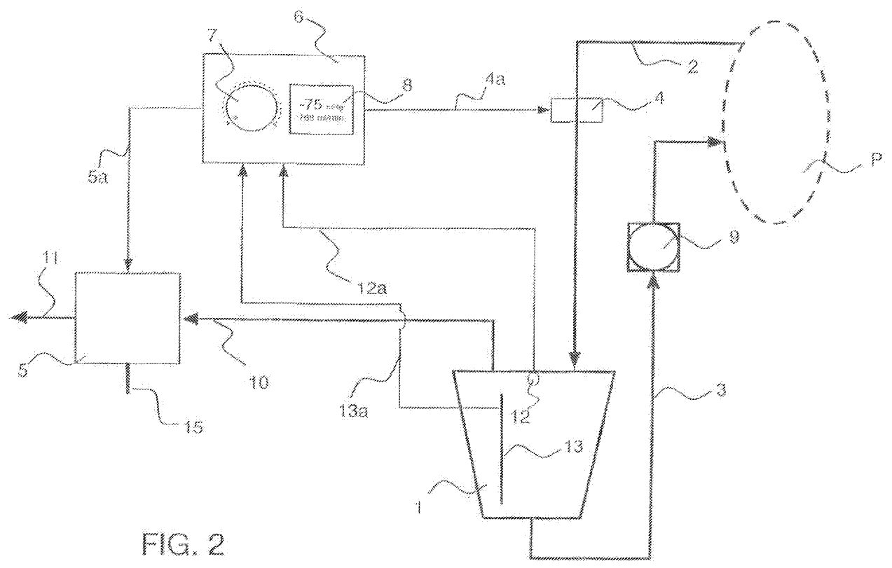

FIG. 2 shows a second embodiment of a device according to the invention, which includes all of the aspects of the first embodiment, and thus reference can be made to the description of the first embodiment in connection with FIG. 1 as regards these points. The second embodiment additionally includes a level sensor 13 that detects the level of blood actually present in the reservoir 1 and thus detects the amount of stored blood. The level sensor 13 generates a second measuring signal which is supplied to the control means 6 via a second measuring signal line 13a. With the aid of the second measuring signal, the control means 6 is able to take the amount of blood actually present in the reservoir 1 into account during vacuum regulation so as to increase the vacuum in the reservoir 1 and thus increase the venous inflow to the reservoir 1 if the level of blood in the reservoir 1 sinks below a predetermined limit. The control means 6 according to the second embodiment can furthermore monitor the reaching of a predetermined upper fill level of the reservoir 1 so as to restrict the venous inflow.

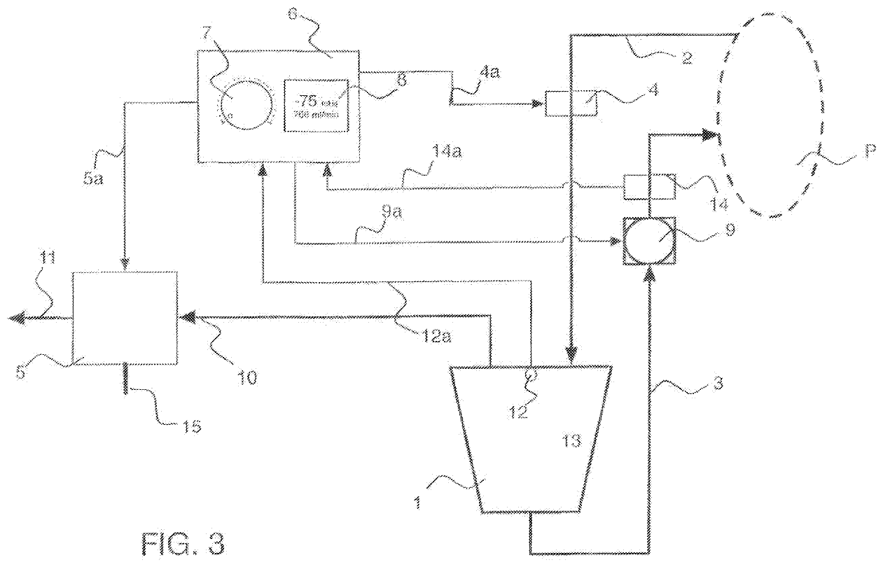

FIG. 3 shows a third embodiment of a device according to the invention, which includes all of the aspects of the first embodiment, and thus reference can be made to the description of the first embodiment in connection with FIG. 1 as regards these points. The third embodiment additionally includes an inflow amount sensor 14 that detects the arterial inflow amount and hence the amount of blood which is supplied to the patient P via the arterial inflow line 3 and is thus removed from the reservoir 1. The amount sensor 14 is arranged on the arterial inflow line 3 either downstream, as shown in FIG. 3, or upstream of the pump 9. The amount sensor 14 outputs a third measuring signal that is supplied to the control means 6 via a third measuring signal line 14a. Owing hereto, the control means 6 is able to take the amount of blood removed from the reservoir 1 into consideration during vacuum regulation and configure the venous inflow accordingly. So as to have an influence on the arterial inflow, the control means 6 in the third embodiment can output a third actuating signal to the pump 9 via a third actuation line 9a, by means of which the delivery rate of the pump is adjusted or at least influenced.

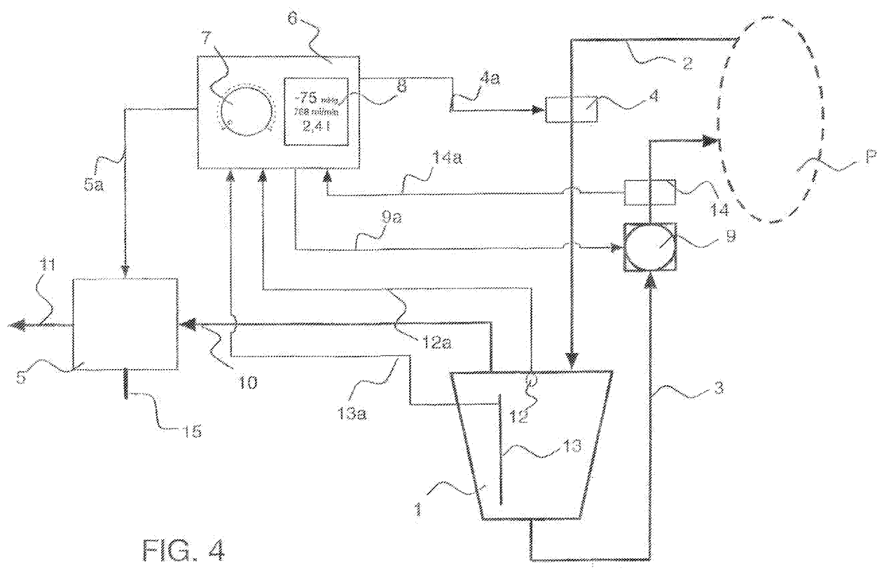

FIG. 4 shows a fourth embodiment of a device according to the invention, which includes all of the aspects of the first three embodiments, and thus reference can be made to the description of the first three embodiments in connection with FIGS. 1, 2 and 3 as regards these points. By combining all of the aspects of the embodiments described above, a device for establishing the venous inflow to the reservoir 1 is created, which allows an almost fully automatic control/regulation of the amount of blood stored in the reservoir 1. The reason for this is that via the gradual closure of the venous inflow line and the regulation of the vacuum in the reservoir 1, the control means 6 can set the amount of blood supplied to the reservoir 1, the current amount of which is respectively detected by the level sensor 13. The control means 6 can at the same time detect the arterial inflow amount via the amount sensor 14 and set or influence it by control of the pump 9. Accordingly, the operating element 7 of the control means 6 can also be configured in the fourth embodiment such that the amount of blood stored in the reservoir 1 is set by the user. This design is taken into consideration in FIG. 4 insofar as the actual fill level of the reservoir 1 is also shown on the display 8. On the basis of the specification of the amount of blood by the user and taking into consideration the physiological marginal conditions that ensure supply of the patient, the control means 6 then effects in a fully automatic manner the adjustment of the venous inflow to the reservoir 1 and the arterial outflow from the reservoir 1 of the extracorporeal blood circulation system.

As is shown in FIGS. 1 to 4, the vacuum unit 5 can be equipped in all of the embodiments with a safety device 15 which, upon corresponding actuation by the control means 6 or if control should fail, interrupts the application of a vacuum to the reservoir 1 and establishes atmospheric pressure in the reservoir 1.

We claim:

1. A device for establishing venous inflow to a blood reservoir of an extracorporeal blood circulation system including a venous inflow line from a patient to the blood reservoir and an arterial inflow line extending from the blood reservoir to the patient, the device comprising:
a restricting unit configured for restricting the venous inflow line;
a vacuum unit configured for applying a vacuum to the blood reservoir; and
a control device that includes only one single operating element for controlling both the restricting unit and the vacuum unit, the single operating element configured to be manipulated in a single manipulation by a user.

2. The device of claim 1, wherein the control device is configured to control the restricting unit and the vacuum unit without the use of a blood level sensor and blood level signals.

3. The device of claim 1, further comprising a level sensor configured to detect a level of blood in the blood reservoir.

4. The device of claim 1, further comprising an inflow amount sensor configured to detect an amount of arterial inflow supplied to the patient.

5. The device of claim 1, wherein the control device further comprises a display device for visual display of a display value corresponding to the venous inflow amount.

6. The device of claim 1, wherein the vacuum unit is connected to a vacuum source via a line.

7. The device of claim 1, wherein the vacuum unit comprises an integrated vacuum source.

8. The device of claim 1, further comprising a vacuum sensor for detecting the vacuum in the blood reservoir, the vacuum sensor connected to the control device for providing a first measuring signal.

9. The device of claim 1, further comprising a blood pump disposed along the arterial inflow line, to which an actuating signal corresponding to a delivery rate of the blood pump established by the user is supplied by the control device to the blood pump, wherein the delivery rate controls a rate at which blood is pumped from the blood reservoir to the patient.

10. The device of claim 1, wherein the single operating element has a plurality of operating positions and the single manipulation establishes both a degree of restriction of the venous inflow line and an amount of vacuum in the blood reservoir for venous inflow to the blood reservoir at, above or below a basic value established by gravitational conveyance.

11. The device of claim 10, wherein at each operating position of the plurality of operating positions, the control device is configured to supply a different combination of a restricting unit setting that corresponds to the operating position to the restricting unit for establishing the degree of restriction of the venous inflow line to define an extent of a restriction of the venous inflow amount to the blood reservoir based on a manipulation of the single operating element in the single manipulation, and a vacuum unit setting that corresponds to the operating position to the vacuum unit for establishing the amount of vacuum in the blood reservoir to define an extent of the increase in the venous inflow amount to the blood reservoir based on the manipulation of the single operating element in the single manipulation.

12. A device for establishing venous inflow to a blood reservoir of an extracorporeal blood circulation system including a venous inflow line from a patient to the blood reservoir and an arterial inflow line extending from the blood reservoir to the patient, the device comprising:
a restricting unit configured for restricting the venous inflow line;
a vacuum unit configured for applying a vacuum to the blood reservoir;
a control device including only one single operating element for controlling both the restricting unit and the vacuum unit, the single operating element having a plurality of operating positions and configured to be manipulated in a single manipulation by a user; and
an inflow amount sensor configured to detect an amount of arterial inflow supplied to the patient.

13. The device of claim 12, further comprising a blood pump disposed along the arterial inflow line, to which an actuating signal corresponding to a delivery rate of the blood pump established by the user is supplied by the control device to the blood pump, wherein the delivery rate controls a rate at which blood is pumped from the blood reservoir to the patient.

14. The device of claim 12, wherein the control device further comprises a display device for visual display of a display value corresponding to the venous inflow amount.

15. The device of claim 12, wherein the vacuum unit comprises an integrated vacuum source.

16. The device of claim 12, further comprising a vacuum sensor for detecting the vacuum in the blood reservoir, the vacuum sensor connected to the control device for providing a first measuring signal.

17. The device of claim 12, wherein the control device is configured to control the venous inflow to the blood reservoir and an amount of vacuum in the blood reservoir without the use of a blood level sensor and blood level signals.

18. The device of claim 12, further comprising a level sensor configured to detect a level of blood in the blood reservoir.

19. The device of claim 12, wherein the vacuum unit further comprises a safety device configured such that, upon actuation by the control device or upon failure of the control device, an application of the vacuum to the blood reservoir is interrupted which establishes atmospheric pressure in the blood reservoir.

20. A device for establishing venous inflow to a blood reservoir of an extracorporeal blood circulation system including a venous inflow line from a patient to the blood reservoir and an arterial inflow line extending from the blood reservoir to the patient, the device comprising:
a restricting unit configured for restricting the venous inflow line;
a vacuum unit configured for applying a vacuum to the blood reservoir;
a control device that includes only one single operating element for controlling both the restricting unit and the vacuum unit, the single operating element having a plurality of operating positions and configured to be manipulated in a single manipulation by a user; and
a safety device configured such that, upon actuation by the control device or upon failure of the control device, an application of the vacuum to the blood reservoir is interrupted which establishes atmospheric pressure in the blood reservoir.
